(19) United States
(12) Patent Application Publication
(10) Pub. No.: US 2004/0106682 A1
(43) Pub. Date: Jun. 3, 2004

(54) Novel benzylideneamino guanidines and their uses as ligands to the melnocortin receptors

(76) Inventors: Torbjorn Lundstedt, Uppsala (SE); Per Andersson, Sollentuna (SE); Arne Boman, Uppsala (SE); Elisabeth Seifert, Uppsala (SE); Anna Skottner, Ekero (SE)

Correspondence Address: Bacon & Thomas, 4th Floor, 625 Slaters Lane, Alexandria, VA, 22314-1176, US

(21) Appl. No.: 10/472,767

(22) PCT Filed: Apr. 5, 2002
(86) PCT No.: PCT/GB02/01593

(30) Foreign Application Priority Data
Apr. 5, 2001 (GB) 0108631.3

(51) Int. Cl. A61K 31/155; C07C 279/26

(52) U.S. Cl. 514/635; 564/234

(57) ABSTRACT

The present invention relates to novel benzylideneamino guanidines of general formula and to the use of these benzylideneamino guanidines as melanocortin receptor agonists or antagonists. The invention further relates to benzylideneamino guanidines which show selectivity to the MC1 and MC4 melanocortin receptors as agonists and/or antagonists.

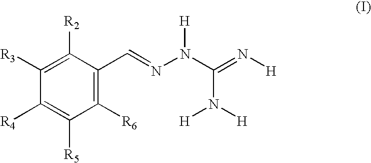

The present invention relates to novel benzylideneamino guanidines. More particularly, it relates to benzylideneamino guanidines that act on melanocortin receptors and to their uses as melanocortin receptor agonists or antagonists. It further relates to these novel benzylideneamino guanidines which show selectivity to the MC1 and MC4 melanocortin receptors as agonists and/or antagonists.

A number of large linear and cyclic peptides are known in the art which show high specific binding to melanocortin (MC) receptors. The agonistic and/or antagonistic properties of these peptides are also known. See for example “Melanocortin Receptor ligands and methods of using same” by Dooley, Girten and Houghten (WO 99/21571). Two patent applications (WO 99/55679 and WO 99/64002) have been published which include small molecules showing activity on the melanocortin receptors. However, the compounds in the present application are structurally different from the previously published melanocortin agonists, and hence the observed effects are unexpected.

Previously known in the art are hydroxyguanidines (e.g. W098/23267), which have proven activity against xanthine oxidase/xanthine dehydrogenase enzymes. Other compounds known in the art are benzylideneamino guanidines which have shown anti-depressive effects (U.S. Pat. No. 4,060,640). Other examples of pharmacologically active guanidines known in the art are described in patent U.S. Pat. No. 3,982,020 and GB1223491. Other application areas are also known in the art and are described in patents DE1165013, and U.S. Pat. No. 3,941,825. Guanabenz is a compound which is well known in the art as an antihypertensive drug (US Pharmacopeia, 1999, The United States Pharmacopeial Convention, Inc., ISBN 1-889788-03-1). Whilst Guanabenz might appear to be structurally similar to the compounds in the present invention, it shows no affinity to the melanocortin receptors. Therefore it is very surprising that the benzylideneamino guanidine compounds in the present invention show affinity to the melanocortin receptors as agonist and/or antagonists.

One aspect of the present invention is therefore to provide low molecular weight compounds showing activity on melanocortin receptors and which may be taken up after per oral administration and which may penetrate well through the blood brain barrier.

In one aspect, the present invention provides novel compounds of the general formula (I):

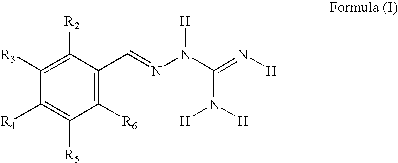

wherein R₂ is selected from halogen, hydroxy, methyl, methoxy or nitro group;

wherein R₃ is selected from a hydrogen, hydroxy, fluoro, chloro or trifluoromethyl group;

wherein R₄ is selected from a hydrogen, nitro, iodo or bromo group;

wherein R₅ is selected from a hydrogen, fluoro or ethoxy group;

wherein R₆ is selected from a hydrogen, nitro, bromo or methoxy group;

and provided that

at least one of R₃, R₄, R₅, R₆ is not a hydrogen;

and when R₄, R₅ and R₆ are hydrogen, then R₂ is selected from a fluoro, bromo, iodo, hydroxy, methyl, methoxy or nitro group;

and when R₄ is a nitro group, then R₂ is selected from a halogen, methyl or methoxy group;

and when R₃ is a fluoro group, then R₂ is selected from a halogen, methyl, methoxy or nitro group;

The invention also extends to the pharmacologically active salts of compounds of formula I.

In the present context, the term “halogen” refers to fluoro, chloro, bromo or iodo.

Preferably, R₆ is hydrogen.

Preferably, R₅ is hydrogen.

Preferably R₂ is a halogen, more preferably bromo or iodo, and most preferably R₂ is iodo.

Preferably R₃ is chloro.

In the present invention the following novel compounds and uses of these compounds are provided:

| No. | Name | Salt | M.p. |
|---|---|---|---|
| 1:1 | N-(3-Chloro-2- | AcOH | 231-232.5 |
| | Iodobenzylideneamino)guanidine | | |
| 1:2 | N-(3-Fluro-2-Methyl-6- | HCl | 189-191 |
| | Nitrobenzylideneamino)guanidine | | |
| 1:3 | N-(2-Chloro-3-Hydroxy-4- | HCl | (decomp.) |
| | Nitrobenzylideneamino)guanidine | | |
| 1:4 | N-(2,3-Dichloro-4- | HCl | 258-262 |
| | Nitrobenzylideneamino)guanidine | | (sublim.) |
| 1:5 | N-(5-Ethoxy-2-Nitro-3- | HCl | 92-98 |
| | Trifluoromethylbenzylideneamino) | | |
| | guanidine | | |
| 1:6 | N-(2,6- | HCl | 178-183 |
| | Dibromobenzylideneamino)guanidine | | |
| 1:9 | N-(2-Bromo-4- | HCl | 236-239 |
| | Iodobenzylideneamino)guanidine | | |
| 1:10 | N-(2,5- | AcOH | 211-212 |
| | Difluorobenzylideneamino)guanidine | | |
| 1:11 | N-(2,6- | AcOH | 158-160 |
| | Dimethoxybenzylideneamino)guanidine | | |
| 1:12 | N-(2,3- | AcOH | 223-224.5 |
| | Dihydroxybenzylideneamino)guanidine | | |
| 1:13 | N-(4-Bromo-2- | AcOH | 202-207 |
| | iodobenzylideneamino)guanidine | | |

M.p. = Melting point in ° C.

To our surprise the above compounds showed activity against the melanocortin receptors.

The present invention relates to novel benzylideneamino guanidines and the use of benzylideneamino guanidines with activity on the melanocortin receptors. The compounds of the present invention have been biologically tested in the melanocortin system and have surprisingly been shown to be capable of binding to melanocortin receptors as well as showing activity in functional assays.

The compounds of the present invention may either be agonists or antagonists of a specific MC-receptor or of a number of MC-receptors, e.g. MC1, MC3, MC4 or/and MC5 receptors.

The MC-receptors belong to the class of G-protein coupled receptors which are all built from a single polypeptide forming 7 transmembrane domains. Five such receptors types, termed MC1, MC2, MC3, MC4 and MC5, have been described. The MC receptor's signalling is mainly mediated via cAMP but also other signal transduction pathways are known. They are distinctly distributed in the body.

MC-receptors are linked to a variety of physiological actions that are thought to be mediated by distinct subtypes of the MC-receptors. In many cases, however, it is not entirely clear which of the subtypes is responsible for the effect.

Some of the compounds provided in the present invention can be used for modulating melanocortin related systems and therefore used for the treatment of diseases such as drug abuse, feeding disorders, immunomodulatory action, pain, skin and sexual function/dysfunctions associated with the melanocortin receptors or related systems, e.g. the melanocyte stimulating hormones.

It has long been known that MSH-peptides may affect many different processes such as motivation, learning, memory, behaviour, inflammation, body temperature, pain perception, blood pressure, heart rate, vascular tone, brain blood flow, nerve growth, placental development, aldosterone synthesis and release, thyroxin release, spermatogenesis, ovarian weight, prolactin and FSH secretion, uterine bleeding in women, sebum and pheromone secretion, blood glucose levels, intrauterine foetal growth, as well as other events surrounding parturition (Eberle, AN: The melanotropins: Chemistry, physiology and mechanisms of action. Basel: Karger, Switzerland. 1988, ISBN 3-8055-4678-5; Gruber, and Callahan, Am. J. Physiol. 1989, 257, R681-R694; De Wildt et al., J. Cardiovascular Pharmacology. 1995, 25, 898-905), as well as inducing natriuresis (Lin et al., Hypertension. 1987, 10, 619-627).

Some of the compounds of the invention are useful for inhibiting or stimulating the in vivo formation of second messenger elements such as cAMP. Such inhibition/stimulation may be used in cells or crushed cell systems in vitro, e.g. for analytical or diagnostic purposes.

For analytical and diagnostic purposes the compounds of the invention may be used in radioactive form where they comprise one or more radioactive labels or gamma or positron emitting isotopes, to be used in radioligand binding for the quantification as well as tissue localisation of MC-receptors, for analysis of dissociation/association constants, and for imaging of in vivo binding by the use of scintigraphy, positron emission tomography (PET) or single photon emission computed tomography (SPECT), or for the diagnosis of disease and treatment of any malignancy where the malignant cells contain MC receptors.

Alternatively the compounds of the invention can be labelled with any other type of label that allows detection of the respective compound, e.g. fluorescence, biotin, or labels activated by gamma-irradiation, light photons or biochemical processes, or by light or UV-light (the latter in order to obtain a compound useful for covalent labelling of MC receptors by a photoaffinity technique).

Some of the compounds of formula (I) or the pharmacologically acceptable salts thereof may also be tagged with a toxic agent (i.e. doxorubicin, ricin, diphtheria toxin or other) and used for targeted delivery to malignant cells bearing MC receptors, or tagged with a compound capable of activating the endogenous immune system for triggering the immune system (for example a compound, monoclonal antibody or other, capable of binding to a T-cell antigen, e.g. CD3 or other) for treatment of malignancies and other MC receptor expressing diseases. The thus formed hybrid compound will direct cytotoxic cells to the malignant melanoma cells or the MC1-receptor bearing malignant cells and inhibit the tumor growth.

Compounds of formula (I) or a pharmacologically acceptable salt thereof may be attached to the antibody chemically by covalent or non-covalent bond(s).

Compounds of the invention may be used for the treatment and diagnosis of diseases, disorders and/or pathological conditions in an animal, in particular in man.

The present invention also relates to a pro-drug which, upon administration to an animal or a human, is converted to a compound of the invention. Pro-drugs of the compounds of formula (I) and their pharmacologically acceptable salts may be used for the same purposes as described in this specification for the compounds of the invention as well as is disclosed in the examples given below.

The compounds of the present invention may be bound covalently or non-covalently to one or several of other molecule(s) of any desired structure(s); the thus formed modified compound or complex may be used for the same purposes as described in this specification for the compounds of the invention as well as is disclosed in the examples given below. In a particularly important embodiment of the invention, a radioactively-labelled molecule is covalently bound to a compound of formula (I) or a pharmacologically acceptable salt thereof so as to make a compound of formula (I) or a pharmacologically acceptable salt thereof radioactively labelled.

The invention also relates to methods for the manufacture and pharmaceutical preparations comprising one or more of the compounds of the invention, as well as to their uses for various medical and veterinary practices related to melanocyte stimulating hormone receptors.

### Methods of Preparation

The invention further provides processes for the preparation of the compounds of formula (I). The compounds may be prepared by the following general method:

Method 1.

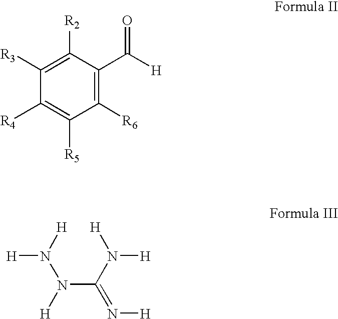

A compound of formula II wherein R₂, R₃, R₄, R₅ and R₆, are as previously defined, is reacted with aminoguanidine (III) or a salt thereof and a compound of formula (I) is obtained.

Many of the benzaldehyde starting materials of the general formula II are commercially available and these and others may also be prepared by any conventional method well known in the art.

### EXAMPLES

The following examples are intended to illustrate but not to limit the scope of the invention, although the compounds named are of particular interest for the intended purposes. These compounds have been designated by a number code, a:b, where a means the number of example, wherein the preparation of the compound is described, and b refers to the order of the compound prepared according to that example. Thus Example 1:2 means the second compound prepared according to Example 1.

The structures of the compounds were confirmed by IR, NMR, MS and elementary analysis. When melting points are given, these are uncorrected.

### Example 1

Preparation of compound 1:1

N-(3-Chloro-2-Iodobenzylideneamino)guanidine

A solution of 3-Chloro-2-Iodo-benzaldehyde (0.32 g, 1.2 mmol), aminoguanidine bicarbonate (0.15 g, 1.1 mmol) and acetic acid (3 ml) was heated at reflux for 5 min. The reaction mixture was cooled down to room temperature and the solution was evaporated. To the residue 50 ml of ether was added and the solution was stirred for 20 min. The solvent was decanted and thereafter 20 ml of acetonitrile was added. The solution was stirred for another 30 minutes and thereafter the solution was filtered, giving the title compound 1:1 with a yield of 0.18 g (42%) M.p. 231-232.5° C.

Preparation of Compounds 1:2-1:13

Compounds 1:2-1:13 were prepared using essentially the same approach as for 1:1 by using Method 1. Compounds with their data was as follows:

| No. | Name | Salt | M.p. |
|---|---|---|---|
| 1:1 | N-(3-Chloro-2- | AcOH | 231-232.5 |
| | Iodobenzylideneamino)guanidine | | |
| 1:2 | N-(3-Fluro-2-Methyl-6- | HCl | 189-191 |
| | Nitrobenzylideneamino)guanidine | | |
| 1:3 | N-(2-Chloro-3-Hydroxy-4- | HCl | (decomp.) |
| | Nitrobenzylideneamino)guanidine | | |
| 1:4 | N-(2,3-Dichloro-4- | HCl | 258-262 |
| | Nitrobenzylideneamino)guanidine | | (sublim.) |
| 1:5 | N-(5-Ethoxy-2-Nitro-3- | HCl | 92-98 |
| | Trifluoromethylbenzylideneamino) | | |
| | guanidine | | |
| 1:6 | N-(2,6- | HCl | 178-183 |
| | Dibromobenzylideneamino)guanidine | | |
| 1:9 | N-(2-Bromo-4- | HCl | 236-239 |
| | Iodobenzylideneamino)guanidine | | |
| 1:10 | N-(2,5- | AcOH | 211-212 |
| | Difluorobenzylideneamino)guanidine | | |
| 1:11 | N-(2,6- | AcOH | 158-160 |
| | Dimethoxybenzylideneamino)guanidine | | |
| 1:12 | N-(2,3- | AcOH | 223-224.5 |
| | Dihydroxybenzylideneamino)guanidine | | |
| 1:13 | N-(4-Bromo-2- | AcOH | 202-207 |
| | iodobenzylideneamino)guanidine | | |

M.p. = Melting point in ° C.

### Example 2

This example illustrates the potency of compounds of formula (I) and their therapeutically active acid addition salts.

Test 1. Affinity for the MC1-receptor

The binding assay was carried out essentially as described by Lunec et al., Melanoma Res. 1992; 2; 5-12 using I¹²⁵-NDP-αMSH as ligand.

Test 2. Affinity for the MC3-receptors, the MC4receptors and the MC5-receptors

The binding assays were carried out essentially as described by Szardenings et al., J. Biol. Chem. 1997; 272; 27943-27948 and Schiöth et al., FEBS Lett. 1997; 410; 223-228 using I¹²⁵-NDP-αMSH as ligand.

Essentially, the affinity of the compounds for the different melanocortin receptors were determined by using either insect cells (Sf9) or COS cells, which were transfected with recombinant human MC3, MC4 or MC5 receptors. For the determination of the affinity for the MC1 receptor, B16 mouse melanoma cells were used, which endogenously express the (mouse) MC1 receptor.

The compounds were tested at different concentrations for their ability to displace a ¹²⁵I-labelled NDP-MSH from the respective receptor. Incubation was performed in 96-well plates, using 50,000 cells/well (Sf9 or COS cells) up to 200,000 cells/well (mouse melanoma cells).

The test compound or standard (NDP-MSH) was added in an appropriate concentration (generally between 10⁻⁴ M and 10⁻¹² M) together with labelled tracer (approx. 50,000 cpm/well) and incubated for 2 hours (at room temperature for Sf9 cells and at +37° C. for COS cells and mouse melanoma cells).

After the incubation, the cells were washed twice to get rid of the excess tracer and compound, and the cells were lysed with 0.1 M NaOH. The lysate was counted in a gamma-counter, binding was calculated and the affinity determined.

TABLE 1
Affinity for MC-receptors.

Ki (μM)

| Compound | MC1 | MC3 | MC4 | MC5 |
|---|---|---|---|---|
| 1:1 | 0.5 | 5.8 | 0.01 | 4.9 |
| 1:4 | 3.6 | 10.1 | 12.2 | 10.7 |
| 1:6 | 0.8 | 22.7 | 0.1 | 29.6 |
| Guanobenz | nb | nb | nb | nb |

nb = non-binding, i.e. no affinity.

### Example 3

In vivo Effects on Food Intake

Compounds have been tested for their effects on food intake and body weight in rats. In order to investigate the agonistic effect, i.e. decrease in food intake, of compounds, the nocturnal protocol was used.

Sprague-Dawley, male rats were used, which were cannulated intracerebroventricularly. Stainless steel guide cannulae were placed in the lateral ventricle and fixed in the skull. Animals were acclimatized for a week before the experiments took place. After the experiments were done, the rats were killed and placement of the cannulae were checked.

Nocturnal Protocol:

Rats were cannulated as described above. They were used without prior starvation, and compounds were administered at 5 pm in a total volume of 5 μl. Doses of compounds used were in between 0.25 to 50 nmoles. Food intake was measured at 3, 15 and 24 hours after dosing, and body weight was recorded at 24 hours. For comparison, the well-known MC4 receptor agonist, Melanotan II (MTII) was used, at a dose of 1 nmole.

### Example 4

Anti Inflammatory Effects

Control

Female BALB/c mice (weight 20-22 g) were sensitized by treatment of the shaved abdomen with 30 μl of 0.5% 2,4-dinitrofluorobenzene (DNFB). After 4 days they were challenged with 10 μl of 0.3% DNFB to the paw. The unchallenged mice paws served as a control. Twenty-four hours after the last challenge, the differences in paws weight were determined as an indicator of the inflammation (paw edema).

Alpha-MSH and Prednisolone Controls

Mice were treated as the control but were additionally injected i.p. with α-MSH (0.5 mg/kg) or prednisolone (20 mg/kg) two hours before sensitization (day 0) and the same dose was administered repeatedly after sensitization during four consecutive days. The paw edema inhibition was measured as described above.

Study of New Compounds

Mice were treated as the control but were additionally injected i.p. with various doses (0.05, 0.15 or 0.25, 0.375, 0.5, 0.75 and in later studies also 1.5, 3 and occasionally 6 mg/kg) of each compound two hours before sensitization (day 0) and the same dose was administered repeatedly after sensitization during four consecutive days. The paw edema inhibition as described above.

Groups containing at least 10 mice each were used for all experiments.

Blood analysis was carried out using the QBC® Autoread™ Plus & QBC® Accutube System (Becton Dickinson). In all cases blood samples were collected twenty-four hours after the last challenge.

### Example 5

Example of a Preparation Comprising a Capsule

| | Per capsule |
|---|---|
| Active ingredient, as salt | 5 mg |
| Lactose | 250 mg |
| Starch | 120 mg |
| Magnesium stearate | 5 mg |
| Total up to | 385 mg |

In cases higher amounts of active ingredient are required, the amount of lactose used may be reduced.

Example of a Suitable Tablet Formulation.

| | Per tablet |
|---|---|
| Active ingredient, as salt | 5 mg |
| Potato starch | 90 mg |
| Colloidal Silica | 10 mg |
| Talc | 20 mg |
| Magnesium stearate | 2 mg |
| 5% aqueous solution of gelatine | 25 mg |
| Total up to | 385 mg |

A solution for parenteral administration by injection can be prepared in an aqueous solution of a water-soluble pharmaceutically acceptable acid addition salt of the active substance preferably in a concentration of 0.1% to about 5% by weight. These solutions may also contain stabilising agents and/or buffering agents.

1. A compound of general formula (I): 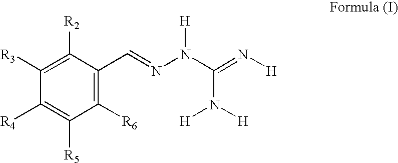
wherein R₂ is selected from a halogen, hydroxy, methyl, methoxy or nitro group;
wherein R₃ is selected from a hydrogen, hydroxy, fluoro, chloro or trifluoromethyl group;
wherein R₄ is selected from a hydrogen, nitro, iodo or bromo group;
wherein R₅ is selected from a hydrogen, fluoro or ethoxy group;
wherein R₆ is selected from a hydrogen, nitro, bromo or methoxy group;
and provided that
at least one of R₃, R₄, R₅, R₆ is not a hydrogen;
and when R₄, R₅, and R₆ are hydrogen, then R₂ is selected from fluoro, bromo, iodo, hydroxy, methyl, methoxy or nitro group,
and when R₄ is nitro, then R₂ is selected from a halogen, methyl or methoxy group,
and when R₃ is a fluoro, then R₂ is selected from a halogen, methyl, methoxy or nitro group,
or a pharmacologically active salt thereof.

2. A compound as claimed in claim 1 wherein R₆ is hydrogen.

3. A compound according to any of the previous claims wherein R₅ is hydrogen.

4. A compound according to any of the previous claims wherein R₂ is bromo or iodo.

5. A compound according to claim 4 wherein R₂ is iodo.

6. A compound according to any of the previous claims wherein R₃ is chloro.

7. A novel compound being any one of the following:
Name
N-(3-Chloro-2-Iodobenzylideneamino)guanidine
N-(3-Fluro-2-Methyl-6-Nitrobenzylideneamino)guanidine
N-(2-Chloro-3-Hydroxy-4-Nitrobenzylideneamino)guanidine
N-(2,3-Dichloro-4-Nitrobenzylideneamino)guanidine
N-(5-Ethoxy-2-Nitro-3-Trifluoromethylbenzylideneamino)guanidine
N-(2,6-Dibromobenzylideneamino)guanidine
N-(2-Bromo-4-Iodobenzylideneamino)guanidine
N-(2,5-Difluorobenzylideneamino)guanidine
N-(2,6-Dimethoxybenzylideneamino)guanidine
N-(2,3-Dihydroxybenzylideneamino)guanidine
N-(4-Bromo-2-iodobenzylideneamino)guanidine
or a pharmacologically active salt thereof.

8. A compound as claimed in any of the previous claims which additionally comprises a label or a toxic agent.

9. A compound as claimed in claim 8 wherein said label is a radioactive label.

10. A pharmaceutical composition comprising a compound as defined in any one of claims 1 to 9, together with one or more adjuvants, carriers or excipients.

11. A process for the production of a compound as claimed in claim 1 which comprises reacting a compound of formula (II) with a compound of formula (III) or a salt thereof 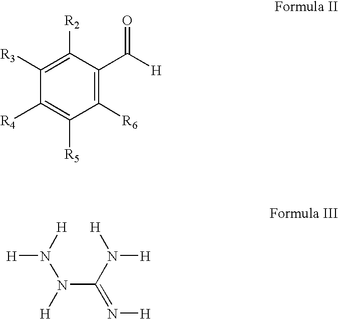
wherein R₂, R₃, R₄, R₅, R₆ are as defined in claim 1, followed by formation if desired of a salt thereof.

12. A compound as claimed in any one of claims 1 to 9 for use as a medicament.

13. A compound according to claim 12 for the treatment of diseases, disorders and/or pathological conditions related to the melanocortin receptors and/or α-MSH or related systems.

14. A compound according to claim 12 for the in vivo diagnosis of diseases, disorders and/or pathological conditions related to the melanocortin receptors and/or (α-MSH or related systems.

15. A method of treating diseases, disorders and/or pathological conditions related to the melanocortin receptors and/or α-MSH or related systems in a subject which comprises administering to said subject an effective amount of a compound according to any one of claims 1 to 9 or a composition as claimed in claim 10.

16. A method of in vivo diagnosis of diseases, disorders and/or pathological conditions related to the melanocortin receptors and/or α-MSH or related systems comprising the use or administration of a compound as defined in any one of claims 1 to 9 or a composition as claimed in claim 10.

17. Use of a compound as defined in any one of claims 1 to 9 for the manufacture of a medicament for treating diseases, disorders and/or pathological conditions related to the melanocortin receptors and/or (α-MSH or related systems.

18. Use of a compound as defined in any one of claims 1 to 9 for the manufacture of a medicament for the in vivo diagnosis of diseases, disorders and/or pathological conditions, related to the melanocortin receptors and/or α-MSH or related systems.
